(19) United States
(12) Patent Application Publication
(10) Pub. No.: US 2005/0250746 A1
(43) Pub. Date: Nov. 10, 2005

(54) Premenstrual dysphoric disorder medication

(76) Inventor: Matthew Iammatteo, Morristown, NJ (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/840,131

(22) Filed: May 6, 2004

(51) Int. Cl. A61K 31/56

(52) U.S. Cl. 514/170

(57) ABSTRACT

A medication to treat the symptoms of premenstrual syndrome and similar conditions related to the periodic change in the physiological level of hormones in a woman's body.

### RELATED APPLICATIONS

None.

### GOVERNMENT INTEREST

None.

### BACKGROUND

Premenstrual Syndrome

Premenstrual syndrome exists when a woman complains of regularly-recurring psychological or somatic symptoms, or both, which occur specifically during the luteal phase of the menstrual cycle and which resolve by the onset of, or during, menstruation. Mild psychological symptoms occur in approximately 95% of all women of reproductive age and can be managed by conservative lifestyle changes. However, for approximately five percent of symptomatic women, the symptoms are so severe that their lives are completely disrupted during the second half of the cycle.

The range of possible symptoms associated with premenstrual syndrome is wide. Premenstrual syndrome (PMS) is diagnosed by symptoms including, most commonly, abdominal bloating, abdominal discomfort and pain, mastalgia (breast pain) and breast swelling, irritability, mood swings, headache, weight gain, fatigue, food cravings, tension, exacerbation of chronic illnesses such as asthma, allergies, epilepsy or migraine. The most commonly reported behavioral or emotional symptoms are dysphoria, irritability, anxiety, tension, aggression, and feelings of being unable to cope and of having a sense of loss of control. It is these latter, emotional or mood impairments which often prompt women to seek medical intervention. Symptoms appear after ovulation, about day 14 of a woman's cycle, and disappear two weeks later as a woman's period starts. The exact cause of premenstrual dysphoric disorder is not completely understood, and the art continues to search for an effective treatment.

Treatments

The etiology of he condition remains unclear and speculative, although many hypotheses have been advanced. This uncertainty in the pathogenesis of premenstrual dysphoric disorder has led to many treatments being suggested as possible therapies for premenstrual dysphoric disorder. As there is a substantial placebo response, however, a large number of uncontrolled clinical trials have resulted in a proliferation of claims for ineffective therapies.

Many available treatments address the superficial physical symptoms, by providing an analgesic pain reliever, or a diuretic. While these products address certain specific symptoms such as headache or weight gain, they do not claim to address the more important emotional-impairment aspects of the condition. The American College of Obstetricians and Gynecologists recommends “lifestyle changes such as aerobic exercise, a complex carbohydrate diet, and/or nutritional supplements such as calcium, magnesium, and vitamin E to help resolve PMS symptoms.” ACOG Issues Guidelines On Diagnosis and Treatment of PMS (31 Mar. 2000) (The American College of Obstetricians and Gynecologists, Washington D.C., publ.).

The puzzle of how to treat the emotional-impairment aspects of premenstrual dysphoric disorder has generated two theories regarding the causes of the emotional-impairment aspects of premenstrual dysphoric disorder. One theory has postulated that premenstrual dysphoric disorder symptoms are caused by changes in the levels of serotonin, a brain chemical. Another theory has postulated that the hormones progesterone, estrogen and testosterone are involved. These two theories have undergone a significant amount of investigation and testing.

Serotonin Levels

For example, serotonin-regulating pharmaceuticals and progesterone have each been tested for use against premenstrual dysphoric disorder. For example, Ellen W. FREEMAN et al., A Double-Blind Trial of Oral Progesterone, Alprazolam and Placebo in Treatment of Severe Premenstrual Syndrome, 274 JAMA 343 (5 Jul. 1995), compares the effectiveness of Alprazolam or progesterone on premenstrual dysphoric disorder symptoms. FREEMAN et al. find that alpazolam shows a 50% reduction in “daily symptom report scores: the authors thus conclude that, “Alprazolam has a role in PMS treatment.” Ellen W. FREEMAN et al., Continuous or Intermittent Dosing With Sertraline for Patients With Severe Premenstrual Syndrome or Premenstrual Dysphoric Disorder, 161 Am. J. Psychiatry 343 (February 2004) reports, “seratraline groups improved significantly more than the placebo group.” (note that this recent publication does not qualify as prior art) Similarly, the American College of Obstetricians and Gynecologists report, “Serotonin selective reuptake inhibitor (SSRIs) antidepressants have been shown effective and may be useful for severe PMS.” ACOG Issues Guidelines . . . , supra. See generally, Katrina M. WYATT et al., Selective Serotonin Reuptake Inhibitors for Premenstrual Syndrome, 1 COCHRANE DATABASE OF SYSTEMATIC REVIEWS 2004 (first published 2002, Issue No. 4).

Progesterone Levels

In contrast, the art has found progesterone treatment ineffective. Katrina WYATT et al., Efficacy of Progesterone and Progestens in Management of Premenstrual Syndrome, 323 BMJ 776 (2001), report “no clinically important difference between progesterone and placebo.” Similarly, Ellen W. FREEMAN et al., supra, found that “progesterone therapy was no better than placebo” and “progesterone is ineffective for PMS.” Similarly, the American College of Obstetricians and Gynecologists conclude, “Oral contraceptives have been widely prescribed as a treatment for PMS, but there is little data to support their effectiveness.” ACOG Issues Guidelines . . . , supra.

### BRIEF DESCRIPTION

In contrast to the prior art, which teaches that premenstrual dysphoric disorder is caused by either a problem with serotonin levels or a problem with hormone levels, I have found that the constellation of symptoms can be treated most effectively by addressing both facets jointly. In contrast to the prior art, which has focused on evaluating progesterone as a premenstrual dysphoric disorder therapeutic (and which teaches that progesterone is not effective), I have tested estrogen as a premenstrual dysphoric disorder therapeutic, and found it provides several advantages.

In contrast to the prior art, I have tested the administration of selective serotonin reuptake inhibitors together with estrogen, or with estrogen and progesterone together, for the treatment of the symptoms of moderate to severe premenstrual syndrome, and have found that it is much more efficacious if these components are delivered together, preferably in the same pill, or transdermal skin patch, or intra-vaginal ring, than if either is administered alone.

### DETAILED DESCRIPTION

I will discuss the various facets of my invention in greater detail.

Premenstrual Dysphoric Disorder

I use the term “premenstrual dysphoric disorder” to encompass the variety of clinical syndromes which are associated with the menstrual cycle and which disrupt the patient's emotional well-being. Thus, I use the term to encompass premenstrual dysphoric disorder (sometimes called late luteal premenstrual dysphoric disorder, premenstrual mastalgia, cyclical mastalgia, premenstrual depression, premenstrual tension or premenstrual dysphoria), the severe, predominately psychological end of premenstrual dysphoric disorder, as well as classical premenstrual syndrome where the psychological or mood impairment plays a significant role.

Luteal phase begins at about day eighteen of the menstrual cycle and ends around day 2 of the following cycle, including taper. This is the window where premenstrual dysphoric disorder has the greatest impact on the patient's emotional health.

SSR Pharmaceuticals

Selective serotonin reuptake inhibitors are known to be effective against moderate to severe depression. A variety of selective serotonin re-uptake inhibitors are known in the art. These include, for example, fluoxetine (commercially available from Eli Lilly & Co., Indianapolis, Ind.), paroxetine (commercially available from the Smith Kline Beecham division of Glaxo Inc., Philadelphia, Pa.), sertraline hydrochloride (commercially available from Invicta Company), fluvoxamine (commercially available from Solvay Corporation), citalopram (commercially available from E.I. du Pont de Nemours & Co., Wilmington, Del.) and alprazolam.

The mechanism of action of SSRI's on premenstrual dysphoric disorder is unknown, as is the precise aetiology of premenstrual dysphoric disorder. Several studies have indicated a serotonergic disturbance in premenstrual dysphoric disorder. Data supporting this includes premenstrual serotonin abnormalities, decreased imipramine platelet binding, luteal phase carbohydrate craving, a blunted prolactin response to tryptophan, increased platelet monoamine oxidase activity premenstrually, and characteristic responses to the serotonin antagonists buspirone and meta-chlorophenylpiperazine. premenstrual dysphoric disorder has similarities to serotonin deficient affective disorder symptoms, such as depression, anxiety, aggression, appetite disturbance and irritability. Additionally, animal models have demonstrated alterations in neurotransmission and neuroreception by ovarian hormones. The serotonin disturbance postulated in premenstrual dysphoric disorder appears to be distinct from that in affective disorders such as depression. This is evident, for example, in the time to clinical efficacy after commencement of treatment.

Decreased libido (sexual desire) and anorgasmia (inability to attain orgasm) are commonly-reported side effects associated with selective serotonin reductase inhibitors. This effect may be exacerbated by the reduction in libido which occurs naturally in the luteal phase of the menstrual cycle in some women. These side effects have been documented as a major persistent complaint in studies using constant (rather than periodic, as, for example, only during the luteal phase) administration of selective serotonin reductase inhibitors to treat clinical depression.

The response of premenstrual dysphoric disorder symptoms to SSRI dosing regimes varies from patient to patient, depending in part on the severity of the patient's premenstrual dysphoric disorder symptoms. Compared to the dosage required for systemic affective disorders, treating premenstrual dysphoric disorder symptoms requires a relatively low dosage. For example, an amount of sertraline effective to treat premenstrual dysphoric disorder can range from less than 50 mg per day to more than 150 mg day. Similarly, fluoxetine is effective at less than 20 mg per day to over 60 mg per day.

At higher daily doses, however, there is an increased incidence of adverse side effects, without a concomitant increase in therapeutic efficacy. To the contrary, individual poor responders do not increase their response at higher dosages, but higher dosages do increase adverse side effects. Interestingly, the serum level of the SSRI does not correlate with therapeutic response.

The effectiveness remains acceptable if the selective serotonin reductase inhibitor is administered in the luteal phase only, rather than continuously through the entire cycle. As luteal-phase only administration reduces adverse side effects, I prefer luteal-phase administration.

Alprazolam, known as an anxiolytic, is effective, yet may pose adverse side effects and possible addiction. Alprazolam is effective if administered in the luteal phase only. Such periodic administration may avoid the adverse side effects associated with constant administration. An amount of alprazolam effective for this use is about 1.5 mg per day, administered orally four times per day. Transdermal administration should reduce the amount required, by avoiding “first-pass” liver metabolism and degradation of the drug substance.

Notably, tricyclic antidepressant compounds such as desipramine hydrochloride do not work as well as SSR compounds in my invention. This is because, in part, treatment of premenstrual symptoms with antidepressants is somewhat successful clinically, but suffers from low rates of acceptance among patients due to sedating side effects. Thus, while they may be considered as legal equivalents of SSR compounds, I do not prefer them.

Estrogen

Estrogen is known in the art. So is its use in “hormone replacement therapy” for women of post-menopausal age, as is its use in contraception, in conjunction with progesterone. We discuss each in turn. The estrogen can be chosen from the group consisting of 17-beta-estradiol, ethynyl estradiol and biocompatible derivatives thereof.

Hormone Replacement Therapy Amount.

For example, Stephen R. CUMMINGS et al., U.S. Pat. No. 6,692,763 (14 Feb. 2004), teaches various low doses of estrogen effective for hormone replacement therapy. CUMMINGS teaches that the amount of exogenous estrogen to be administered to the patient should be sufficient to achieve a serum estradiol level of at least about 5 pg per ml, but not more than about 20 pg per ml, and preferably not more than about 15 pg per ml. In other words, CUMMINGS teaches that sufficient exogenous estrogen is administered to achieve a total serum estradiol level of roughly 5 to 20 pg per ml.

Since the serum estradiol level of an untreated subject will differ for each individual, different individuals may require administration of different doses of estrogen to achieve the required serum estradiol level. Often, the amount of exogenous estrogen to be administered is sufficient to achieve a serum estradiol level of between about 5 pg/ml and about 10 pg/ml. Serum estradiol levels of between 5 pg/ml and 15 pg/ml produce a decrease in the risk of vertebral and hip fracture due to osteoporosis, a condition common among post-menopausal women. While earlier researchers have espoused higher levels of estrogen for hormone replacement therapy, the administration of the lower-than-conventional amount of exogenous estrogen taught by CUMMINGS et al., has the advantage of decreasing the risk of undesirable side effects associated with hormone replacement therapy.

Contraceptive hormones have been widely prescribed as a treatment for premenstrual dysphoric disorder. Despite such wide use, there is little data to support their effectiveness against the emotional symptoms of premenstrual dysphoric disorder.

Contraceptive Amount

Suitable estrogen components for the method of this invention for contraception are the known estrogens. In this connection, the estrogen employed should be administered in such dosages that the amount of estrogen utilized according to this invention is equal to that corresponding to 0.030-0.050 mg of 17.alpha.-ethinylestradiol, as measured in conventional tests. See, e.g., J. UFER, HORMONTHERAPIE IN DER FRAUENHEILKUNDE, at page 27 (De Gruyter Verlag Berlin-New York, publ.) (1972). The 17-α-ethinylestradiol esters and ethers, as well as the estradiol esters (such as 17-α-ethinylestradiol), are suitable as the estrogen component.

The estrogen may be conventional estrogen, or an equivalent (often called a “gestagen”), employed according to this invention in combination with the estrogen. It can be the same or preferably different in the different stages of the menstrual cycle. When different gestagens are utilized in the first and second stages, the side-effects of a specific gestagen are reduced or eliminated by administering a first gestagen in one stage, while another gestagen, which has a competitive behavior with respect to the side-effects, is administered in the other stage. Thus, it is possible, for example, to use the estrogen in one stage in combination with a gestagen derived from testosterone or 19-nortestosterone which optionally has a substituted hydrocarbon residue in the 17-α-position. These (19-nor-) testosterone derivatives generally exhibit a minor androgenic side effect. In the other stage, the estrogen can then be employed in combination with a gestagen derived from progesterone which does not exhibit the androgenic side effect inherent in such testosterone or 19-nortestosterone derivatives. Those derivatives are considered especially advantageous which, in addition to the gestagenic activity, have anti-androgenic side effects.

When using different gestagens in the first and second stages, one can employ, in the first stage of the menstrual cycle, the estrogen in combination with a testosterone or 19-nortestosterone derivative as the gestagen component and, in the second stage, the estrogen in combination with a progesterone derivative as the gestagen component.

Suitable as the gestagen component according to the present invention are all substances having significant gestagenic activity. In this connection, the gestagen employed should be administered at such dosages that the amount of gestagen utilized in the first 10-12 days according to the menstrual cycle corresponds to about 0.050 to about 0.125 mg. daily of d-norgestrel as measured in the conventional tests. See J. UFER, supra, at page 28. The amount of gestagen employed according to this invention in the 11-9 days of the second phase of the menstrual cycle is about 2-3 times that employed in the first phase, i.e., it corresponds in activity to about 0.100 to 0.350 mg. daily of d-norgestrel.

Interestingly, it is known in the art that progesterone is not effective against premenstrual dysphoric disorder. See, e.g., Katrina WYATT, Efficacy of Progesterone and Progestogens in Management of Premenstural Syndrome: A Systematic Review, 323 BMJ 776 (2001) (review of published research concludes that there is “no clinically important difference between progesterone and placebo.”).

Formulation

One can prepare variations of my invention in a variety of forms, such as oral pills or tablets, trans-dermal skin patches, and vaginal inserts.

Oral Dosage Form

One can achieve the benefits of my invention by manufacturing a pharmaceutical composition of matter comprising a selective serotonin reductase inhibitor and a contraceptive effective combination of estrogen and progesterone. For example, a premenstrual medication may be formulated:

| Fluvoxamine | 5 to 25 | mg |
|---|---|---|
| Ethinyl estradiol | 20 to 30 | μg |
| Gestodene | 75 | μg |

The formulation is complete as desired, as, for example, formulated with butylated hydroxyanisole, edentate disodium, hydrogenated soybean oil flakes, hydrogenated vegetable oil, soybean oil and coloring agent. Stabilizer and antioxidant are included as needed to mitigate chemical cross-reaction between the teratogen and the contraceptive.

As another example, an oral premenstrual pharmaceutical composition may be formulated:

| paroxetine | 10 to 25 | mg |
|---|---|---|
| Estrogen | 20-30 | μg |

The formulation is complete as desired, with anhydrous lactose, microcrystalline cellulose, polyvinylpyrrolidone, stearic acid, colloidal anhydrous silica and gelatin. As with the preceding example, stabilizer and antioxidant are useable as needed to mitigate chemical cross-reaction between the teratogen and the contraceptive.

Cross-Reactivity

The incidence of adverse chemical reaction between the SSR pharmaceutical and the estrogen/progesterone components can be avoided in a variety of ways known in the art of pharmaceutical formulation. For example, one or several of the drug substances can be provided as a core of active ingredient, which core is built up or coated with a barrier which remains chemically inert until the product is used. This approach is taught in, for example, DEBREGEAS et al., U.S. Pat. No. 4,960,596. Alternatively, the various drug substances could be mixed with an inert compound which provides an inert matrix separating the various drug substances until ingested. This approach is taught in, for example, U.S. Pat. No. 5,529,791 (25 Jun. 1996).

Transdermal Formulations

One may deliver these compositions using transdermal drug delivery technology. This avoids first-pass liver metabolism, thus reducing the amount of the various active ingredients required to be effective. An example of a transdermal delivery system is a backing layer and an adhesive polymer matrix which has dispersed therein the selective serotonin uptake inhibitor and the hormones effective for controlling fertility, as well as a combination of skin permeation enhancers. As well as providing the matrix within which the hormones and skin permeations are dispersed, the adhesive polymer matrix also serves to adhere the delivery system in intimate contact with the skin of the subject being treated to permit the hormones to be absorbed transdermally.

The materials used for the backing layer can be laminates of polymer films with a metal foil such as aluminum foil. The backing layer can be be a thickness of from about 10 to about 200 microns. The adhesive polymer matrix can be fabricated from biologically-acceptable adhesive polymers, such as polyacrylic adhesive polymers, silicone adhesive polymers or polyisobutylene adhesive polymers, solid and dimensionally stable, but preferably thin, e.g. from about 10 to about 200 microns in thickness.

The adhesive polymer matrix can further include a moisture-regulating humectant or plasticizer dispersed therein. The humectant/plasticizer can be a polyol, such as polyethylene glycol, such as a liquid polyethylene glycol, with a molecular weight of about 200 to about 450. The inclusion of polyethylene glycol serves to control the rigidity of the polymer matrix, as well as acting as a moisture regulating humectant. Incorporation of a humectant in the adhesive polymer matrix allows the TCDS to absorb moisture on the surface of skin, which in turn helps to reduce skin irritation and to prevent the TCDS from falling off during long term (such as 7 days) use of the TCDS. The amount of humectant/plasticizer utilized can range from about 0 to about 25%, but preferably, the amount of humectant/plasticizer utilized will be less than 5%, e.g., about 0.25-2.5% of the total adhesive polymer matrix.

The skin permeation enhancers utilized (if any) could consist of any combination of dimethyl sulfoxide (DMSO), a fatty alcohol ester of lactic acid and lower (C.sub.1-C.sub.4) alkyl ester of lactic acid, such as lauryl lactate (commercially available as CERAPHIL 31™ from Van Dyk Chem. Co., Belleville, N.J.) and ethyl lactate. A combination of skin permeation enhancers, when homogeneously dispersed in an adhesive polymer matrix at a particular ratio (preferably, 2.5-5:1:1, respectively), acts to solubilize the dispersed estrogen and progestin, thus greatly enhancing the skin permeation of the steroid hormones contained in the transdermal patch.

The skin permeation enhancer combination may also enhance the tackiness and adhesion of the transdermal patch.

Optionally, an additional adhesive layer can be formed using the same or a different adhesive polymer which is also biocompatible and placed in intimate contact with the surface of the hormone-containing adhesive polymer layer. This adhesive layer can contain one or more effective transdermal absorption enhancing agents or be free of these agents.

A trans-dermal skin patch using my combinations can achieve the same benefit of minimizing the risk of unwanted pregnancy. For example, one can prepare a trans-dermal hormone replacement therapy daily skin patch as follows:

| Progesterone | 1.5 to 10 | mg |
|---|---|---|
| Sertraline hydrochloride | 10 to 50 | mg |
| Estrogen | 0.3 to 1.25 | mg |
| PDMS-382 (Dow Corning) pre-polymer | 9.2 | gm |
| 5-Amino-5-ethyl-2-(3-haptyl_1,3-dioxane | 500 | mg |
| Polymerization initiator | 1 | drop |

After mixture, the mixture is poured into sheet molds and allowed to set at room temperature for 24 hours. After set-up is complete, the sheet may be cut into discs 1 centimeter in diameter. Other trans-dermal formulations are well within the teachings of the art, and depend on the nature of the specific skin penetrating agent used.

Dosage Amounts

With respect to the dosage amount of the pharmaceutical components used here, the specific dose may vary depending on the age and weight of the patient, the severity of the symptoms, the incidence of non-teratogenic side effects and the like. For isotretinoin, for example, it is known in the art to administer 5-40 milligrams of isotretinoin two times per day. Similarly, for a contraceptive, it is known in the art to use estrogen, 20 or more micrograms per day, or gestodene 75 micrograms per day together with 20-30 micrograms per day of ethinyl estradiol per day. Other currently-known contraceptive preparations are discussed in my Information Disclosure Statement at Shangold et al., U.S. Pat. No. 6,214,815.

I propose to formulate a daily dose of SSRI at doses which deliver from about 10 to about 25 mg per day of SSRI drug substance, as a lower range of therapy (for less severe symptoms), combined with a contraceptive amount (about 0.25 to 0.5 mg of estrogen and 0.15 to 1.0 mg progesterone). This can advantageously be used in the luteal phase of the cycle only. For more severe symptoms, one can use a daily does of from about 20 mg to about 50 mg of the SSRI, combined with a contraceptive amount of hormones (from about 0.25 to about 0.5 mg of estrogen and 0.15 to 1.0 gm of progesterone).

With respect to hormone replacement therapy, I propose to lower and lessen the doses of the SSRI in same distribution as mentioned above with estrogen alone (0.3 to 1.25 gm per day) or, for women lacking ovaries (e.g., post-hysterectomy women), combined with progesterone (1.5 to 10 mg per day).

### CONCLUSION

On reading the forgoing, it will be readily apparent to one of skill in the art to make modifications and variations on my basic concept. I thus intend my patent to cover not just the specific examples I talk about here, but all the material covered by the legal claims appended here, and legal equivalents of these claims.

Note that in the claims, I use certain terms in specific ways, with specific definitions. I use the word “a” to include more than one (i.e., as used in the claims, it means “at least one, and maybe more”).

1. A product for treating premenstrual dysphoric disorder, comprising:
a. a hormone comprising estrogen and progesterone; and
b. a selective serotonin reuptake inhibitor;
c. said estrogen and said progesterone present in amounts which together are effective as a contraceptive; and
d. said selective serotonin reuptake inhibitor present in an amount effective to reduce emotional impairment related to premenstrual dysphoric disorder, when administered with said hormone.

2. The product of claim 1, wherein:
c. said estrogen and said progesterone are present in amounts which together are effective as a contraceptive during luteal phase; and
d. said selective serotonin reuptake inhibitor present in an amount effective to reduce emotional impairment related to premenstrual dysphoric disorder, when administered with said hormone during luteal phase.

3. The product of claim 2, formulated as an oral dosage form.

4. The product of claim 2, formulated as a transdermal skin patch.

5. The product of claim 1, formulated as an intra-vaginal ring.

6. The product of claim 1, formulated as an oral dosage form.

7. The product of claim 1, formulated as a transdermal skin patch.

8. The product of claim 1, formulated as an intra-vaginal ring.

9. A method for treating premenstrual dysphoric disorder, the method comprising administering the product of claim 1 to a premenstrual dysphoric disorder patient.

10. A method for treating premenstrual dysphoric disorder, the method comprising administering the product of claim 2 to a premenstrual dysphoric disorder patient.

11. A method for treating premenstrual dysphoric disorder, the method comprising administering the product of claim 3 to a premenstrual dysphoric disorder patient.

12. A method for treating premenstrual dysphoric disorder, the method comprising administering the product of claim 4 to a premenstrual dysphoric disorder patient.

13. A method for treating premenstrual dysphoric disorder, the method comprising administering the product of claim 5 to a premenstrual dysphoric disorder patient.

14. A method for treating premenstrual dysphoric disorder, the method comprising administering the product of claim 6 to a premenstrual dysphoric disorder patient.

15. A method for treating premenstrual dysphoric disorder, the method comprising administering the product of claim 7 to a premenstrual dysphoric disorder patient.

16. A method for treating premenstrual dysphoric disorder, the method comprising administering the product of claim 8 to a premenstrual dysphoric disorder patient.

17. A product for treating postmenopausal depression, comprising:
a. a hormone comprising estrogen; and
b. a selective serotonin reuptake inhibitor;
c. said estrogen present in hormone replacement therapy-effective amount; and
d. said selective serotonin reuptake inhibitor present in an amount effective to reduce depression in a postmenopausal patient, when administered with said hormone.

18. The product of claim 17, formulated as an oral dosage form.

19. The product of claim 17, formulated as a transdermal skin patch.

20. The product of claim 17, said hormone further comprising progesterone in a hormone replacement therapy-effective amount.

21. A method for treating postmenopausal depression, the method comprising administering the product of claim 17 to a postmenopausal patient.

22. A method for treating postmenopausal depression, the method comprising administering the product of claim 18 to a postmenopausal patient.

23. A method for treating postmenopausal depression, the method comprising administering the product of claim 19 to a postmenopausal patient.

24. A method for treating postmenopausal depression, the method comprising administering the product of claim 20 to a postmenopausal patient.
